(19) United States
(12) Patent Application Publication
(10) Pub. No.: US 2009/0064398 A1
(43) Pub. Date: Mar. 12, 2009

(54) Swimming Goggles with a Frameless Unitary Len

(76) Inventor: Terry CHOU, Talnan City (TW)

Correspondence Address: Dr. BANGER SHIA;Int''l Patent Office of Bang Shia, 102 Lindencrest Ct, Sugar Land, TX, 77479-5201, US

(21) Appl. No.: 11/852,314

(22) Filed: Sep. 9, 2007

(51) Int. Cl. A61F 9/02

(52) U.S. Cl. 2/439; 2/426

(57) ABSTRACT

The present invention discloses a pair of holistically designed swimming goggles, including a unitary len, a len-receiving pad, and a securing strap. The specialized unitary len includes a front-lateral curve, a len-pad engaging rim and a len-strap engaging window. The front-lateral curve is a smooth transition between an up-front view and a lateral view without an angle formed threrein. The len-receiving pad includes a pad-len engaging rim, a pad-len engaging rim extension, and a pad-face contact.

### BACKGROUND OF THE INVENTION

1. Field of the Invention

The present invention relates to a piece of swimming goggles. In particular, the present invention relates to a combination of a single piece of frameless unitary len, a len-receiving pad and a securing strap for a pair of swimming goggles, which comes with an improved assembling convenience, reduced manufacturing cost, a low maintenance demand as well as a substantially broadened view and a streamline holistic design.

2. Description of the Related Art

The manufacturing of most conventional swim goggles requires at least three components: the lens, the frame and the padding, in addition to the securing head strap. In conventional goggles, a frame provides both a securing and a connecting function for an assembly of lens and padding. FIG. 6 of the drawings illustrates a pair of conventional swimming goggles with U.S. Pat. No. 6,804,835, comprising a lens-assembly 10′, a frame 11′, a padding member 12′, and a head securing strap 13′. Detailed description of this prior art is given below.

The lens-assembly 10′ is made of transparent material. Each of lens 101′ (of the lens-assembly 10′) comprises a flange on an outer periphery thereof for engaging with the frame 11′ and the padding member 12′. A central connecting plate 102′ is mounted between the two lens 101′ and comprises at least one engaging hole (two in this embodiment of FIG. 6). Each of lens 101′ is composed of three distinguished views: the up-front view 1011′, the lateral view 1012′, and an annular view 1013′ in between the up-front view 1011′ and the lateral view 1012′. A protruding rim 1014′ is configured at the back of each of lens 101′ to engage with the padding member 12′.

The frame 11′ is manufactured to contain the lens-assembly 10′ within by having a compartment 111′, as well as to engage with the lens-assembly 10′ via a frame-lens connecting plate 112′.

The padding member 12′ is made of rigid material and comprises an pad-lens engaging rim 121′. At the rear of the pad-lens engaging rim 121′ is formed a pad-lens engaging rim extension 122′ and a pad-face contact 123′. As a result, the head securing strap 13′ sits firmly around the rear of a swimmer's head in such a way that the pad-face contact 123′ nestles nicely Around the eye sockets of a goggle wearer's.

Please refer to FIG. 7 and 8 for further understanding of the above-referenced prior art, which requires a frame for lens as a main component. In addition, two lens are utilized in the lens-assembly; two-piece lens structure cost more in manufacture since two symmetric and yet different modes of lens are needed.

### SUMMARY OF THE INVENTION

The goal of the present invention is to provide a pair of swimming goggles with improved assembling convenience, a broadened view, and a streamline appealing look at a reduced manufacturing cost, without affecting its utility durability.

The goal of easy assembling is achieved by dispensing with one of the main components in most conventional goggles, i.e., the frame for lens. Furthermore, a single piece of unitary streamline len replaces the indispensable two pieces of lens in most prior arts, which significantly enlarges a goggle wearer's view at a reduced manufacturing cost, as described below.

The present invention distinguishes itself from related prior arts in that only two main components are utilized: the lens and the padding. The role of a frame in related prior arts is taken over by a single piece of specialized len as well as its shape-matching padding. The single specialized len engages securely with the matching padding, and at the same time, enlarges and improves the visible view by having a smooth curve surface at its lateral view, as compared with the angular lateral view for lens in most prior arts. As a result, the structure of the present invention is substantially simplified and the disadvantage of blocking a swimmer's lateral view by a non-transparent frame is at the same time obviated.

To be more specific, in accordance with an aspect of the invention, a pair of swimming goggles comprises a single piece of frameless unitary len, a len-matching pad and a securing strap. The len is made of transparent material, of which the width corresponds to that of the eyes of a swimmer. The len includes two symmetric up-front views in front of a swimmer's eyes with respect to its center, corresponding to a swimmer's nose position. Close to the two ends of the len is each formed a lateral view. A smoothly curve surface is configured in between the up-front view and the lateral view, the design of which substantially increase the range of a swimmer's view. Along the perimeter of the rear surface of the len is formed a len-pad engaging rim while a len-strap engaging window is formed at each end of the len, neighboring the lateral view.

Other objects, advantages, and novel features of the invention will become more apparent from the following detailed description when taken in conjunction with the accompanying drawings.

### BRIEF DESCRIPTIONS OF THE DRAWINGS

FIG. 1 is an exploded perspective view of a pair of swimming goggles in accordance with the present invention.

FIG. 2 is a front view illustrating assembling of the frame and the lens in FIG. 1.

FIG. 3 illustrates the pair of swimming goggle in FIG. 2 along the vertical A-A cross-sectional view.

FIG. 4 illustrates the pair of swimming goggles in FIG. 2 along the horizontal B-B cross-sectional view.

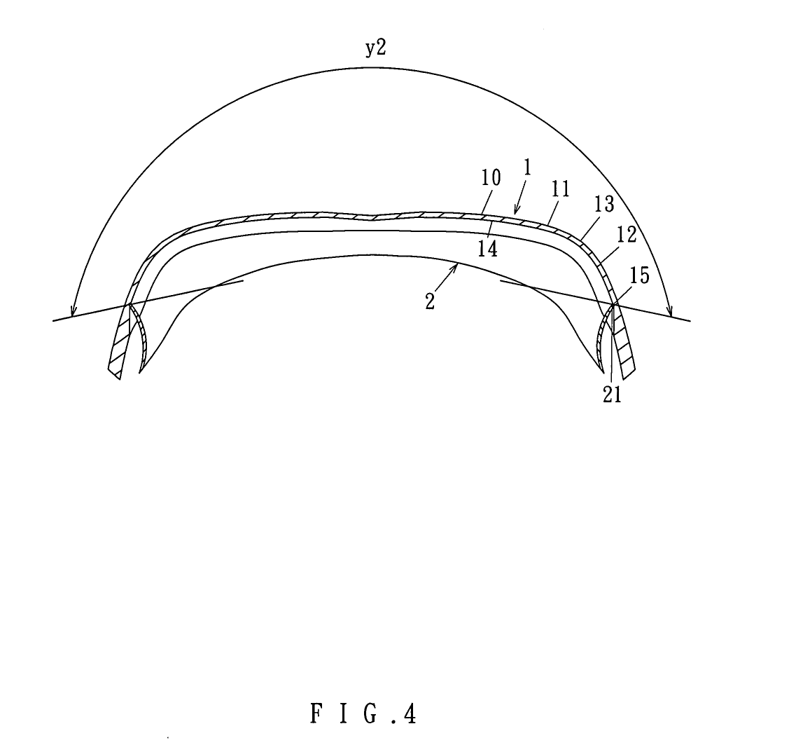

FIG. 5 is an assembly perspective view of a pair of swimming goggles in accordance with the present invention.

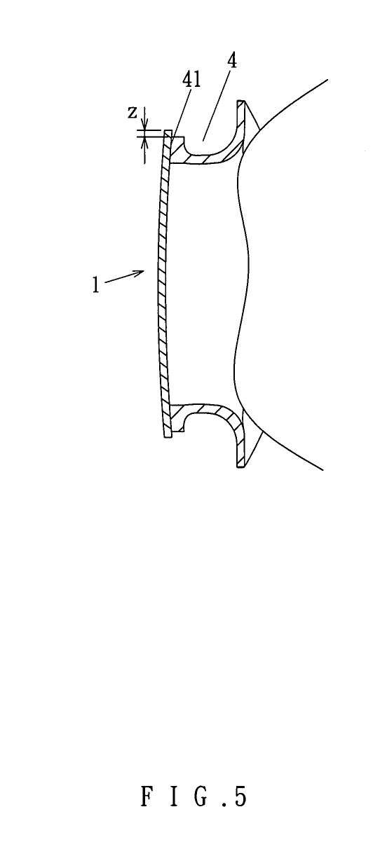

FIG. 6 is an exploded perspective view of a pair of conventional swimming goggles.

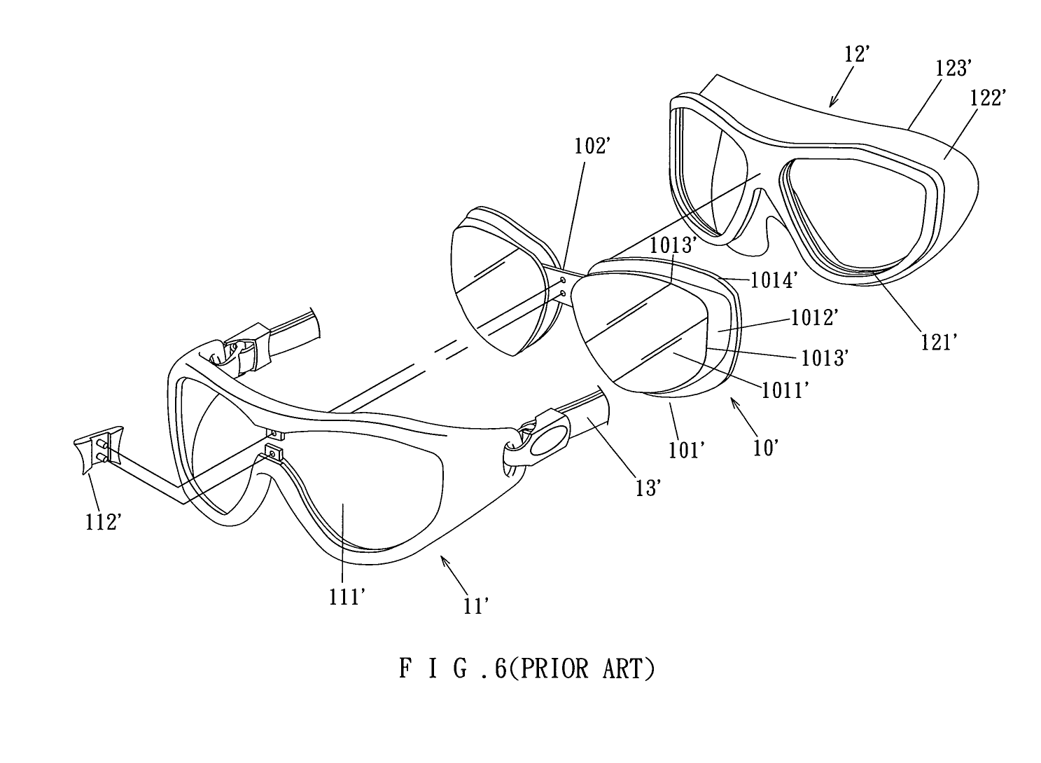

FIG. 7 is an assembly view of the pair of conventional swimming goggles in FIG. 6

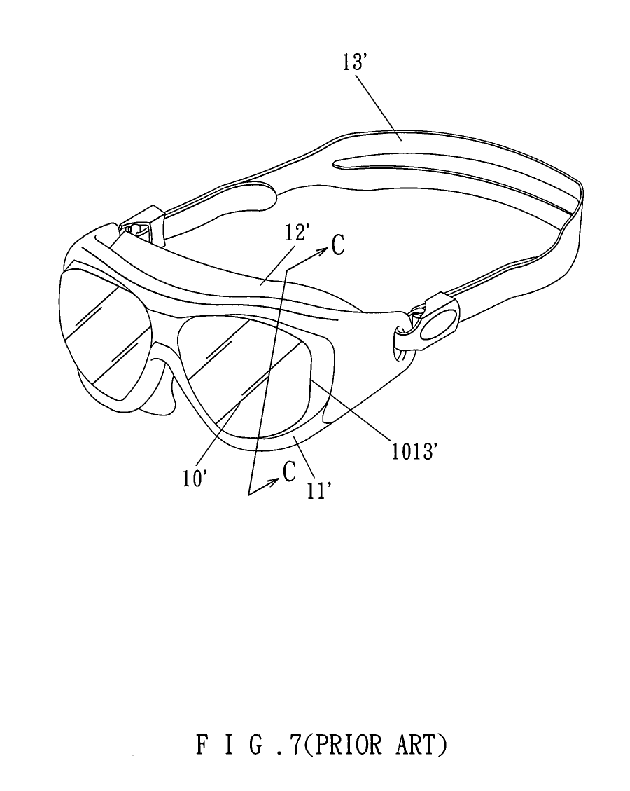

FIG. 8 is an exploded perspective view of a pair of swimming goggles in FIG. 7 along the C-C cross-sectional view.

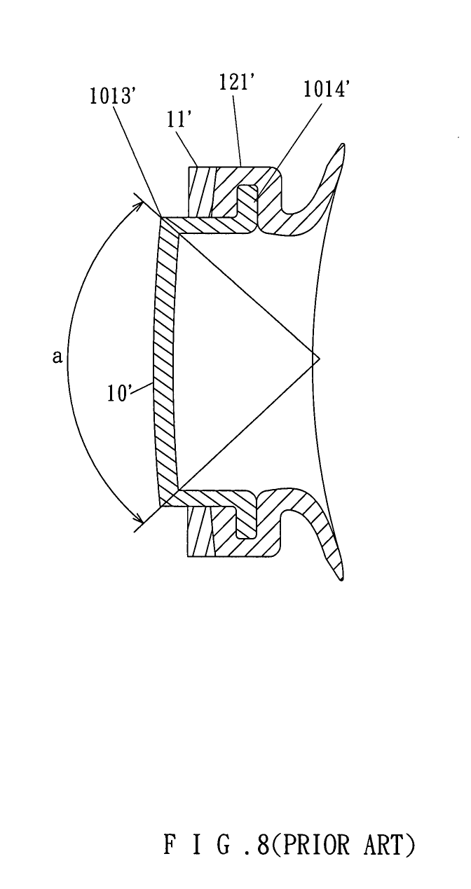

### DETAILED DESCRIPTIONS OF THE DRAWINGS

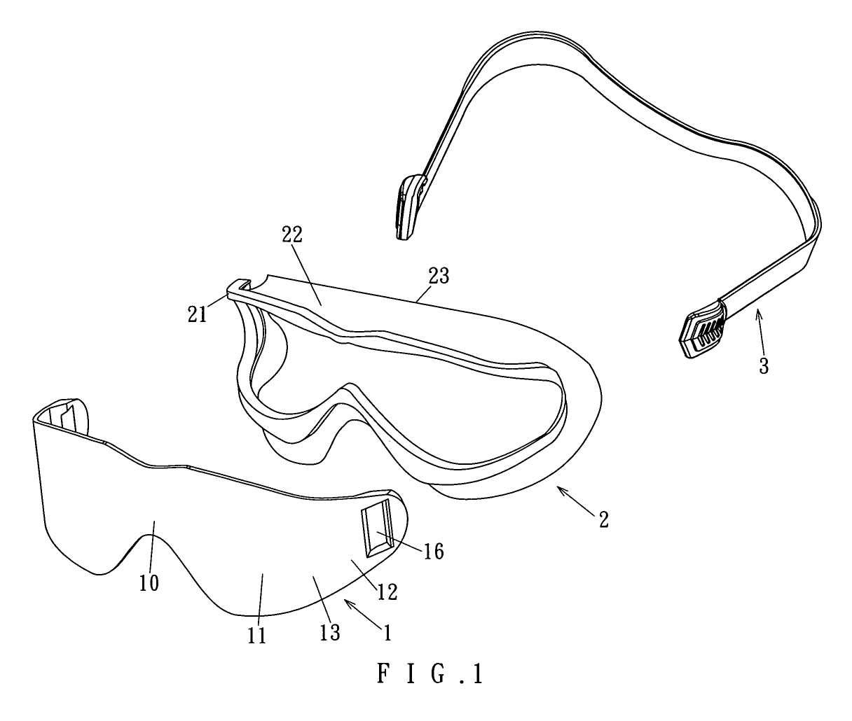

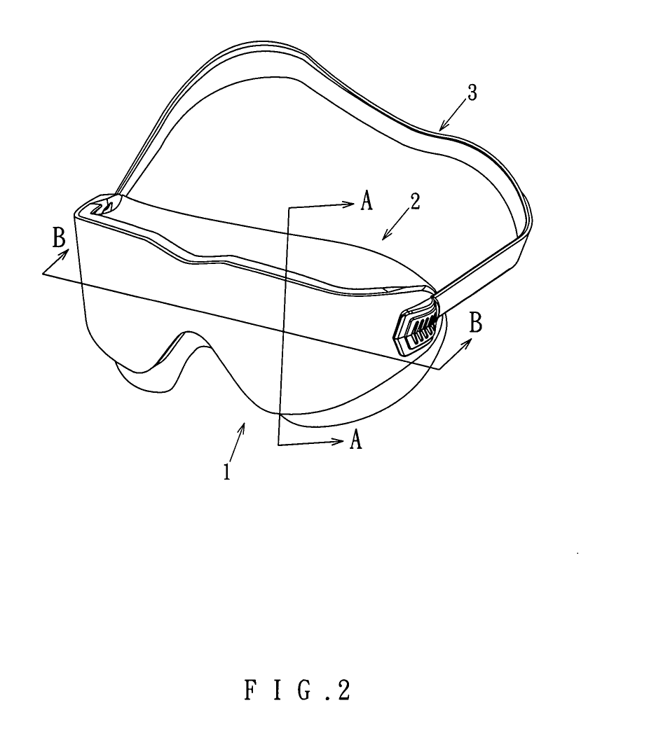

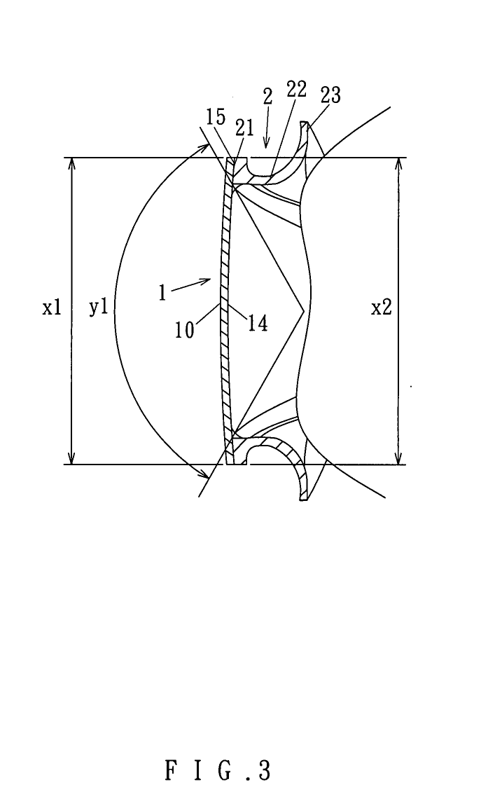

Referring to FIGS. 1 to 3, a piece of swim goggles in accordance with the present invention comprises mainly a single piece of len 1, a len-receiving pad 2, and a securing strap 3. Detail descriptions are given to each of the three components below.

(1) A single unitary len: The len 1 further includes seven sections: the central-front 10, the up-front 11, the lateral view 12, the front-lateral curve 13, the rear surface 14, the len-pad engaging rim 15, and the len-strap engaging window 16. The up-front 11 flanks the central-front 10 (of the len 1); the front-lateral curve 13 is configured in between the up-front 11 and the lateral view 12, whereas the lateral view corresponds in general to a goggle wearer's temple position; the rear surface 14 takes up the surface of the rear of the len 1; a len-pad engaging rim 15 is formed along the perimeter of the rear surface 14 (of the len 1); at either end of the len 1 is configured a len-strap engaging window 16.

(2) A len-receiving pad: The len-receiving pad 2 is designed to fit the len 1 so that the combination of the two comfortably and securely sit around the eyes of a swimmer.

The pad 2 is made of soft materials with the shape of a loop, configured to match that of the len 1. The len-receiving pad 2 includes a pad-len engaging rim 21, a pad-len engaging rim extension 22, and a pad-face contact 23. The pad-len engaging rim 21 engage with the len-pad engaging rim 15 (of the len 1); the pad-len engaging extension 22 extends from the pad-len engaging rim 21; the pad-face contact 23 touches nicely to the face of a goggles wearer.

(3) A securing strap: Each of the two ends of the securing strap 3 connects to the len 1 via its corresponding len-strap engaging window 16 in such a way that the strap fits a swimmer's forehead. As a result, the len-matching pad 2 nestles closely to the eye sockets of a swimmer.

Although specific embodiments have been illustrated and described, numerous modifications and variations are still possible without departing from the essence of the invention. The scope of the invention is limited by the accompanying claims.

What is claimed is:

1. A pair of swimming goggles, comprising three components:
(I) a unitary len, made of transparent material, of which the width corresponds to that of eyes of a goggle wearer, comprising:
(1) a central-front view, physically corresponding to a nose position,
(2) an up-front view, flanked on either side of said central-front view,
(3) a lateral view, located on either side of said unitary len,
(4) a front-lateral curve, which is a smooth transition between said up-front view and said lateral view without containing an angle formed threrein,
(5) a rear surface, along the perimeter of which is configured a len-pad engaging rim, and
(6) a len-strap engaging window, formed at either end of said unitary len following said lateral view;
(II) a len-receiving pad, made of soft materials and in a loop-like shape to physically correspond to said unitary len, comprising:
(1) a pad-len engaging rim, configured to interlock with said len-pad engaging rim of said rear surface of said unitary len,
(2) a pad-len engaging rim extension, which extends from said pad-len engaging rim, and
(3) a pad-face contact, which neighbors said pad-len engaging rim extension; and
(III) a securing strap, the two ends of which connect to those of said unitary len respectively via said len-strap engaging window.

2. The pair of swimming goggles as claimed in claim 1, wherein the area of said pad-len engaging rim is configured to be less than or equal to that of the perimeter of said unitary len to avoid the former being protruding out of the latter and become visible after assemblying.

3. The pair of swimming goggles as claimed in claim 1, wherein said pad-len engaging rim interlocks with said len-pad engaging rim by gluing.

4. The pair of swimming goggles as claimed in claim 1, wherein said pad-len engaging rim interlocks with said len-pad engaging rim in manufacturing.
